(19) United States
(12) Patent Application Publication
(10) Pub. No.: US 2007/0265505 A1
(43) Pub. Date: Nov. 15, 2007

(54) Apparatus and Method For Providing a Controlled Environment Around the Eyes

(75) Inventors: Michel Guillon, London (GB); Cecile Adrienne Maissa, London (GB)

Correspondence Address: SENNIGER POWERS, ONE METROPOLITAN SQUARE, 16TH FLOOR, ST LOUIS, MO, 63102, US

(73) Assignee: Michel Guillon, London (GB)

(21) Appl. No.: 10/596,639

(22) PCT Filed: Dec. 20, 2004
(86) PCT No.: PCT/GB04/05354

§ 371 Date: May 30, 2007

(30) Foreign Application Priority Data
Dec. 19, 2003 (GB) 0329510.2

(51) Int. Cl. A61B 3/10

(52) U.S. Cl. 600/300; 351/205

(57) ABSTRACT

Apparatus which provides a dry environment around the eyes of the user comprising: an eye enclosure adapted to provide an enclosed area about the eyes of the user; means for retaining the eye enclosure in position; and means for supplying dry air to the eye enclosure. Also described are apparatus and methods for testing items to be applied to the eye, screening patients, diagnosing ocular and/or systemic medical conditions, and treating ocular and/or systemic medical conditions.

The present invention relates to testing apparatus and methods. In particular it relates to apparatus for providing a controlled environment, in the region of the eye of the user. In a further arrangement, it relates to an apparatus and method for testing contact lens materials, contact lens care products and medicaments for use in the eye. More particularly it relates to testing apparatus for evaluating eye drops for use in the treatment of dry eye symptoms. In a further embodiment the present invention relates to a method and apparatus for use in the screening of patients and/or to the diagnosis of patients having dry eye symptoms. In a still further embodiment the present invention relates to apparatus for use in the treatment of patients suffering from dry eye symptoms and a method of treating patients suffering from dry eye symptoms. In a still further embodiment the present invention relates to apparatus and method for diagnosing ocular and/or systemic medical conditions.

“Dry eye” which is more correctly known as dry eye syndrome is defined as a disorder of the tear film of the eye which is either caused by the eye not producing sufficient tears to perform their functions of bathing the eye, washing out dust and debris and keeping the eyes moist or by excessive tear evaporation due to the inefficient chemical composition of the tears. Symptoms include persistent feelings of dryness, itching and burning feelings. Some patients complain of experiencing the feeling of a foreign body being in the eye. Surprisingly, watery eyes may also be a symptom of dry eye syndrome. This is because the excessive dryness over stimulates the watery component of the tears but the composition of the tears is such that evaporation occurs rapidly and the dry eye problems are not alleviated.

The prevalence of dry eye syndromes has been estimated by Schein O D et al in “Prevalence of dry eye among the elderly” Am J Ophthalmol. 1997; 124:723.728 to be as high as 4.3 millions in the USA for adults between the age of 65 and 84. In a more recent study, Moss S E et al “Prevalence and risk factors for dry eye syndrome” Arch Opthalmol. 2000; 118: 1264-1268 which was carried out on a larger cohort population between the ages of 48 and 91, reported a 14.4% prevalence. Further the Canadian Dry Eye Epidemiology Study, which surveyed patients across all age groups, reported that 28.7% experienced dry eye symptoms, of these 7.8% indicated constant but moderate symptoms and 1.6% severe symptoms. This was reported in Doughty M J et al “A patient questionnaire approach of dry eye symptoms in patients presenting to optometric practices across Canada” Optom. Vis. Sci 1997:74(8):624-631. Additionally an increased prevalence of dry eye with age in older woman has been observed by McCarty C A et al “The epidemiology of dry eye in Melbourne, Australia” Opthalmology 1998: 105;1114-1119.

Dry eye syndrome may be caused by several factors. For example for some patients it is part of the natural aging process. It is particularly prevalent in women during the menopause. As dry eye syndrome is most common in women it is postulated that it may be associated with fluctuations in hormone levels. Taking medications such as antihistamines, antidepressants and contraception pills may also cause dry eye syndrome. It may also be a symptom of systemic diseases such as lupus, rosacea or Sjogren's syndrome (in which the patient experiences dry eyes, dry mouth and rheumatoid arthritis or lupus). External factors such as climate or contact lens wear may also result in dry eye syndrome. Further causes of the syndrome include incomplete closure of the eyelids, eyelid disease and a deficiency of the tear-producing glands.

Currently dry eye syndrome cannot be cured. However, the symptoms of dryness, itching and burning can be managed. Topically applied artificial tears and lubricants in the forms of eyedrops, gel or ointments, containing viscosity enhancing agents used in various range of concentrations, in preserved or unpreserved formulations, have been shown to help in the relief of the symptoms present in mild dry eye conditions, with more viscous products being used to address the more pronounced symptoms. In more severe cases, Restasis cyclosporine ophthalmic emulsion eyedrops may be prescribed which help to increase the natural production of tears. Another proposed solution is the application of temporary collagen punctal plugs or permanent silicone punctal plugs in the tear ducts which act to prevent the natural tears from draining away too quickly.

In order to correctly diagnose dry eye syndrome it is often necessary to place the patient in a dry atmosphere to see how their eyes react. To achieve this dry rooms are created which are rooms in which the humidity and temperature is controlled such that the ambient air is dry i.e. at a humidity of less than about 40% and usually less than about 20%. For the patient assessments the humidity will generally be in the range of from about 5% to about 30%. However, dry rooms used in the manufacturing of moisture sensitive or hygroscopic products can provide less than about 1% relative humidity.

These rooms may also be used in assessing new types of contact lens, checking a patient's compatibility with contact lenses and for testing materials and products for application to the eye particularly in the treatment of dry eye syndrome. However, these rooms are expensive to set up and maintain and are therefore only available at a few sites. Since the room is at a particular humidity, the range of investigations, tests etc. which can be carried out at any one time may be restricted. Adjusting the humidity may also take a significant time, particularly if the room is large.

A further drawback is that the optometrist, researcher or other health professional may have to enter the room with the patient in order to take measurements or carry out tests and the like. Long term exposure of this kind may be disadvantages to these workers.

It is therefore desirable to provide apparatus and methods which provides a controlled environment in the ocular region and which is relatively inexpensive to produce.

U.S. Pat. No. 6,270,467 and U.S. Pat. No. 6,210,000 describe a system, apparatus and method for preventing Computer Vision Syndrome (CVS). When using computers, many users do not blink as often as is required to maintain a proper preocular tear film. To address this problem, it is suggested that the computer user should wear goggles to which is attached means for providing the user with a blink reminder signal and optionally means to monitor the user's blink rate. In one arrangement the goggles include means for moistening the area enclosed by the goggles. This is achieved by the use of a nebulizer attached to the eye enclosure which is in fluid communication with the enclosed area and is adapted to provide a supply of nebulized air to the enclosed area.

In an alternative arrangement a moistening fluid is applied to the computer user's eyes so that the eyes act as a fluid supply reservoir from which the fluid evaporates to moisten the enclosed area. This embodiment may further comprise a moistening fluid supply, a pump member in fluid communication with the moistening fluid supply and a fluid communication line which is positioned and adapted to direct the moistening fluid to the computer user's eyes.

In a further alternative arrangement, the moistening is provided by means of evaporation of fluid from a reservoir.

Whilst the goggles proposed in these patents make some suggestions as to how increased humidity may be achieved, there is nothing within the disclosure to suggest that a dry atmosphere could or should be achieved. Further, there is no suggestion as to how such a dry atmosphere could be achieved or for what purposes it could be used.

According to the present invention there is provided apparatus which provides a dry environment around the eyes of the user comprising:
an eye enclosure adapted to provide an enclosed area about the eyes of the user;
means for retaining the eye enclosure in position; and
means for supplying dry air to the eye enclosure.

By “dry air” we mean air which has a lower humidity than the ambient air. In particular, dry air will be air having a humidity of about 40% or less. Most preferably the dry air of the present invention will have a humidity of about 30% or less, more preferably 20% or less. In some embodiments it may be as low as from less than 1% to about 5%.

The apparatus may be provided with dry air from, for example, a gas canister. However, the apparatus may include means for generating the dry air. Any suitable means for generating the dry air may be used. Generally ambient air will be passed through a container of a suitable desiccant such as calcium sulphate before being supplied to the eye enclosure. Other suitable desiccants include silica gel, activated alumina, sodium chloride, montmorillonite clay, calcium aluminosilicate clay, silica clay, bentonite clay, titanium silica gel, molecular sieves (sodium alumino-silicates, calcium sodium alumino-silicates, potassium sodium alumino-silicates) activated alumina, lithium chloride and the like. Particularly suitable desiccant units are available under the trade mark DRIERITE and can be purchased from W. A. Hammond Drierite Co. Ltd of P O Box 460, Xenia, Ohio 45385. Air produced in this manner generally has a humidity of less than 1%. A liquid desiccant system may also be used. The container in which the desiccant is placed and through which the air is passed is preferably sealed to prevent the desiccant from absorbing moisture from the environment such that its useful life is prolonged. In one alternative arrangement cold condensation coils may be used to remove the water from the air.

The dry air will generally be pumped to the eye enclosure.

In one alternative arrangement, air may be provided to the eye enclosure from a source of dry air and a source of “wet” air and mixed such that a particularly desired level of dryness can be achieved. “Wet” air is air having a higher humidity than that of ambient air. The wet air is formed by any suitable means but will generally be provided by passing the ambient air through water or through a mixture of, for example, water and glycerol. The air is preferably passed through the water or water/glycerol mixture in a suitable container which will generally be sealed to prevent leakage.

Where mixed dry and wet air is to be used, the apparatus preferably includes means for measuring the relative humidity of the mixed air and means for adjusting the mixture so that the desired level of humidity may be achieved. The measurement means and the adjustment means preferably allow the alteration of the humidity of the air supplied to the eye enclosure to be adjusted during operation. For example, the air supplied may become progressively dryer over, timed intervals or it may start as very dry air and then be increased in humidity. The arrangement also allows air having a humidity greater than that of ambient air to be supplied to the eye enclosure during part of the period of operation.

Whilst the wet and dry air may be supplied separately to the eye enclosure such that mixing occurs within the eye enclosure, it is preferred that the wet and dry air is mixed, for example in a mixing chamber, before being supplied to the eye enclosure.

Means may additionally be included to monitor and/or regulate the flow of air to the eye enclosure.

Where wet and dry air is to be mixed, valves may be present such that the relative amounts of wet and dry air supplied either to the eye enclosure or to the mixing chamber can be adjusted.

Where the humidity of the air in the eye enclosure, or being supplied to the eye enclosure, is to be measured, measurement may be carried out by any suitable means but will generally be by electronic means. In one arrangement, the apparatus provides that where the measurement of the humidity is not the predetermined desired humidity, the flow of wet and/or dry air to either the eye enclosure or the mixing chamber is adjusted automatically by operation of valves.

In a preferred arrangement, the means for supplying dry air to the eye enclosure allows for substantially equal air flow to the region of each eye. This may be achieved by providing a “Y-junction” such that the air stream, which may be a stream of mixed air, is split into two streams and then supplied through two lines one of which connects to the eye enclosure adjacent to the left eye and the other adjacent to the right eye.

The eye enclosure is preferably a pair of goggles. The goggles may be of any suitable configuration. The means of retaining the eye enclosure in position may be arms of the kind used in spectacles which extend such that they can be located behind the ear. In one alternative the eye enclosure may be held in place by a band suitable to be placed around the head. The band may be a single piece, such as of elastic material or two joinable pieces may be used each extending from opposing sides of the eye enclosure. The two bands may be joined by tying or they may include fastening means such as a clip, buckle or Velcro™.

In one arrangement the goggles may be a mask type which provides a chamber covering both eyes. In one alternative arrangement two separate chambers may be present. In this arrangement the goggles may be a swimming goggle type arrangement. Where two chambers are used, each chamber may be supplied with air having a different humidity and thus each chamber may be associated with a respective supply line and in one arrangement separate systems for providing the air to the enclosure.

The eye enclosure when in place on the user's face preferably forms a close fit such that the humidity of the air within the enclosure is not altered by the humidity of the ambient air. However, it is preferably not an air tight fit such that air from within the enclosure can be vented as fresh dry air is supplied to the enclosure to prevent an increase in pressure. In addition the venting will avoid excessive condensation from occurring within the goggles due, for example, to perspiration from the skin.

The enclosure may be made of any suitable material although to minimise the weight of the apparatus it is likely that a plastics material will be used. It is preferred that at least the portion of the eye enclosure which will be located in front of the eyes will be formed from transparent material such that the user can see whilst wearing the apparatus. In an alternative arrangement, at least the portion of the eye enclosure which will be located in front of the eyes in use, will be formed from material through which the eye may be viewed and tested, for example, by an optometrist or other professional.

Where the eye enclosure is configured to allow the eye to be viewed and tested it may be coated with an anti-reflection coating such that optometric instruments used to observe, for example, the tear film, can function or such that vision testing can be carried out. The anti-reflection coating may be provided over the entire surface of the enclosure or over the portion of the enclosure that is central to the eye when the user is looking straight ahead. A hydrophobic coating may additionally or alternatively be included.

The apparatus of the present invention is preferably portable. In one arrangement, the means for supplying air, pump and the like may be placed in a carry case which when not in use will also include the eye enclosure. In use, lines connecting the pump to the eye enclosure will extend from the carry case.

In one arrangement, the apparatus may include means for measuring the temperature of the air which is present in, or supplied to, the eye enclosure. In this arrangement there is preferably also means for adjusting the temperature.

The apparatus of the present invention enables the user to be exposed to a particular chosen level of dry air for the required period of time. The level may be adjusted during treatment and the eyes may be observed.

The apparatus of the present invention is particularly suitable for use in the testing of contact lens materials, contact lens care products, eyedrops or ocular medication.

Thus according to a second aspect of the present invention there is provided a method of testing an item to be applied to the eye comprising:
applying the test item to at least one of the user's eyes;
subjecting the user to an environment around the eyes that is adjusted from the ambient environment by providing an eye enclosure adapted to provide an enclosed area about the eyes of the user and supplying air having the adjusted environment to the eye enclosure;
and monitoring the user.

In an alternative method of the second aspect of the present invention there is provided a method of testing an item to be applied to the eye comprising:
subjecting the user to an environment around the eyes that is adjusted from the ambient environment by providing an eye enclosure adapted to provide an enclosed area about the eyes of the user and supplying air having the adjusted environment to the eye enclosure;
removing the eye enclosure and applying the test item to at least one of the user's eyes; and monitoring the user.

In this alternative arrangement, the eye enclosure may be replaced once the test item has been applied to the at least one of the user's eyes.

In a still further arrangement, the eye enclosure may include means to enable the test item to be introduced without the need for the enclosure to be removed. Thus the enclosure may include a door, or where the test item is, for example, eye drops, they may be passed through a vent or line or even injected through the surface of the enclosure.

The monitoring of the user in the above methods may be carried out periodically or continuously while the user is wearing the eye enclosure or it may be carried out once the subjection of the user to the chosen environment is completed or the user may periodically remove the eye enclosure for monitoring to take place.

One advantage of the present invention is that it is possible to alter the environment within the eye enclosure rapidly.

The adjustment of the environment may be an adjustment of one or more components of the environment including, but not limited to, humidity, temperature or the addition of a foreign bodies such as pollen, pollutants and the like.

The ability to adjust such components rapidly enables the situation of users entering and leaving air conditioned buildings to be simulated.

According to a third aspect of the present invention there is provided testing apparatus for use in testing an item to be applied to the eye comprising:
an eye enclosure adapted to provide an enclosed area around the eyes of the user;
means for retaining the eye enclosure in position; and
means for supplying air to the eye enclosure having an environment that is adjusted from the ambient environment.

The item to be tested may be, for example, contact lens, contact lens material, contact lens care products, eye care products or medicaments. In particular the item to be tested is medicaments, most particularly medicaments for use in the treatment of dry eye syndrome. The medicaments may be in the form of eye drops. Other medicaments may also be tested including those for treating irritations of the eye such as the symptoms of hay fever.

The adjustment of the environment may be an adjustment of one or more components of the environment including, but not limited to, humidity, temperature or the addition of a foreign bodies such as pollen, pollutants and the like.

The eye enclosure adapted to provide an enclosed area about the eyes and to allow for the supply of air having adjusted environment may be the apparatus of the above first aspect of the present invention either providing dry air or adapted to provide alternative environments. The environment provided may be altered during the testing.

In one alternative arrangement, the apparatus used may be that of the above first aspect of the present invention adapted to supply air having a higher humidity than ambient air. As detailed above, the apparatus of the above first aspect may include means for mixing the wet air and dry air and thus in this embodiment the ratio of dry and wet air may be adjusted such that the resultant air has a higher humidity than ambient air. In the alternative, wet air may be provided directly to the eye enclosure.

It is desirable that a reliable method and apparatus is available for use in screening patients to assess their compatibility with, for example, contact lenses in general, specific types of contact lenses, treatment regimes and the like.

Thus according to a fourth aspect of the present invention there is provided a method of screening patients comprising the steps of:
subjecting the user to an environment around the eyes that is adjusted from the ambient environment by providing an eye enclosure adapted to provide an enclosed area about the eyes of the user and supplying air having the adjusted environment to the eye enclosure;
and monitoring the user.

The adjustment of the environment may be an adjustment of one or more components of the environment including, but not limited to, humidity, temperature or the addition of a foreign bodies such as pollen and the like.

According to a fifth aspect of the present invention there is provided screening apparatus for use in screening a patient comprising:
an eye enclosure adapted to provide an enclosed area around the eyes of the user;
means for retaining the eye enclosure in position; and
means for supplying air to the eye enclosure having an environment that is adjusted from the ambient environment.

The adjustment of the environment may be an adjustment of one or more components of the environment including, but not limited to, humidity, temperature or the addition of a foreign bodies such as pollen and the like.

The patient may be being screened for suitability for contact lens wear, compatibility with a particular type of contact lens or treatment regime for example with contact lens care products, eye care products or medicaments.

The method of the second and fourth aspect and the apparatus of the fifth aspect may be used to compare the performance of contact lens materials, contact lens adjunct products, and other ophthalmic products including medicaments, punctal plugs and the like. The benefit achieved is that the operator can be sure that the tests of the materials are carried out at identical conditions since the environment, such as the humidity, can be held at a fixed level, preferably a low level, for a determined period of time. A further benefit achieved is that the materials being tested may be tested to extreme conditions which may the superiority of one product to become apparent.

The eye enclosure adapted to provide an enclosed area about the eyes and to allow for the supply of air having adjusted environment may be the apparatus of the above first aspect of the present invention.

In one alternative arrangement, the apparatus used may be that of the above first aspect of the present invention modified to supply air having a higher humidity than ambient air as detailed above in connection with the above second and third aspects.

According to a sixth aspect of the present invention there is provided a method for diagnosing ocular and/or systemic medical conditions comprising:
subjecting the user to an environment around the eyes that is adjusted from the ambient environment by providing an eye enclosure and adapted to provide an enclosed area about the eyes of the user and supplying air having the adjusted environment to the eye enclosure; and monitoring the user.

The ocular condition may be dry eye syndrome. In the diagnosis of dry eye evaporative problems, it may be useful to subject the user to both an environment that is of higher humidity than ambient atmosphere and an environment of lower humidity than ambient atmosphere.

The monitoring of the user in the above methods of this aspect of the present invention may be carried out periodically or continuously while the user is wearing the eye enclosure or it may be carried out once the subjection of the user to the chosen environment is completed or the user may periodically remove the eye enclosure for monitoring to take place.

The methods may be carried out repeatedly. Each repetition may be carried out at the same or different adjusted environments.

According to a seventh aspect of the present invention there is provided apparatus for use in diagnosing a patient suffering with dry eye syndrome comprising:
an eye enclosure adapted to provide an enclosed area around the eyes of the user;
means for retaining the eye enclosure in position; and
means for supplying air to the eye enclosure having a humidity that is adjusted from the ambient humidity.

The eye enclosure adapted to provide an enclosed area about the eyes and to allow for the supply of air having adjusted humidity may be the apparatus of the above first aspect of the present invention.

In one alternative arrangement, the apparatus used may be that of the above first aspect of the present invention modified to supply air having a higher humidity than ambient air as detailed above in connection with the above second and third aspects.

According to an eighth aspect of the present invention there is provided a method of treatment for patients having ocular and/or systemic medical conditions comprising:
an eye enclosure adapted to provide an enclosed area about the eyes of the user;
means for retaining the eye enclosure in position; and
means for supplying air having a required environment to the eye enclosure.

The required environment may be a required humidity.

The ocular condition may be dry eye syndrome.

According to a ninth aspect of the present invention there is provided treatment apparatus for patients suffering from ocular and/or systemic medical conditions comprising:
an eye enclosure adapted to provide an enclosed area around the eyes of the user;
means for retaining the eye enclosure in position; and
means for supplying air to the eye enclosure having an environment that is adjusted from the ambient environment.

The required environment may be a required humidity.

The eye enclosure adapted to provide an enclosed area about the eyes and to allow for the supply of air having adjusted humidity may be the apparatus of the above first aspect of the present invention.

In one alternative arrangement, the apparatus used may be that of the above first aspect of the present invention modified to supply air having a higher humidity than ambient air as detailed above in connection with the above second and third aspects.

In the eighth and ninth aspect of the present invention, the air provided to the eye enclosure will usually be air having an increased humidity over that of ambient air and will preferably have a humidity of at least 50%

The ocular condition is preferably dry eye syndrome.

The patient may find that maintaining the eye in a relatively high and controlled level of humidity will alleviate symptoms of dry eye, particularly with those experiencing severe symptoms. The patient may also find that the recovery of the ocular surface towards normality will be facilitated.

In one arrangement of the above mentioned aspects of the present invention the eye enclosure may be capable of providing environment for a series of different hygrometry conditions. These may be 5%, 15%, 25% to 30%, 40% to 50% and >60% to 70%. These may be selected to represent specific atmospheric conditions.

5% simulates extreme dryness conditions such as those experienced in some long haul airline flights particularly in the aircraft cockpit or some extreme outdoor conditions such as those experienced in mid-winter in the Canadian prairie.

15% simulates near extreme conditions such as those experienced in the passenger cabin of mid to long haul flights, or in heated buildings during the winter.

25% to 30% simulates low humidity conditions which can be associated with discomfort and are often experienced in offices with air conditioning and/or central heating.

40% to 50% simulates comfortable humidity conditions, recommended residential conditions.

>60% to 70% simulates conditions above normal residential relative humidity or outdoor rainy day conditions in temperate climates.

In the above testing methods, the method may be carried out with several testing sessions at different humidity levels. For example a testing regime may be provided in which some environments supplied are representative of dry conditions and others are representative of high humidity conditions.

The various embodiments of the apparatus of the present invention not only enable various selected environmental conditions to be provided but also to enable the user to conduct various operations which may effect the eye, such as computer use, reading and viewing television. Thus the methods of the present invention can be conducted with the user carrying out set tasks such as those known to affect the eye.

The present invention will now be described by way of example with reference to the accompanying drawings in which:

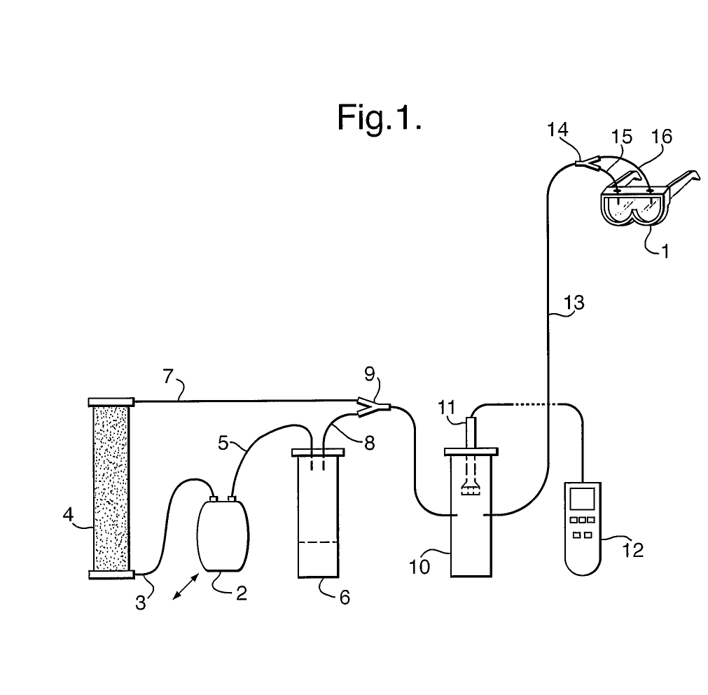

FIG. 1 is a schematic diagram of the apparatus of a preferred embodiment of the present invention;

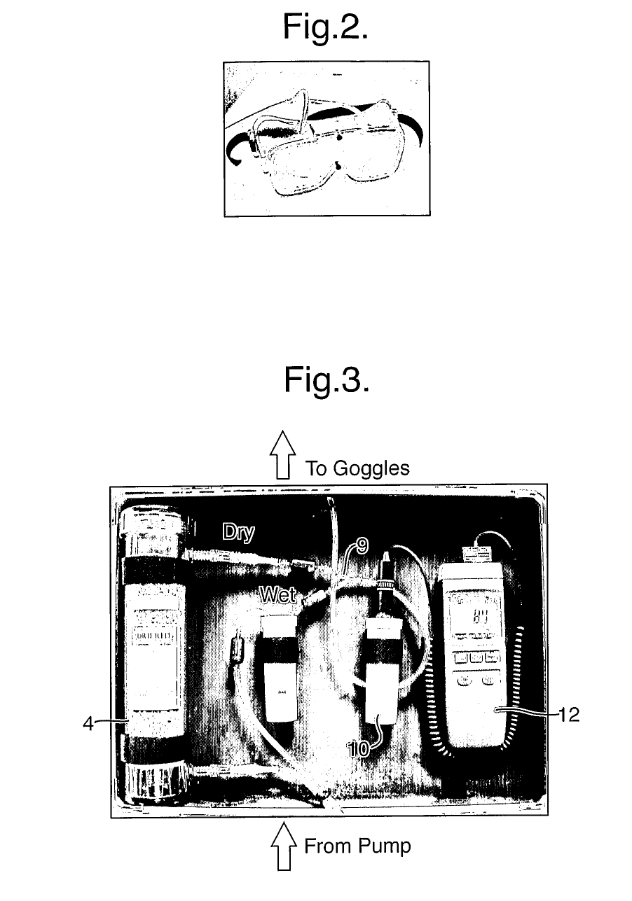

FIG. 2 is a close up picture of an eye enclosure;

FIG. 3 is a picture of a part of prototype arrangement;

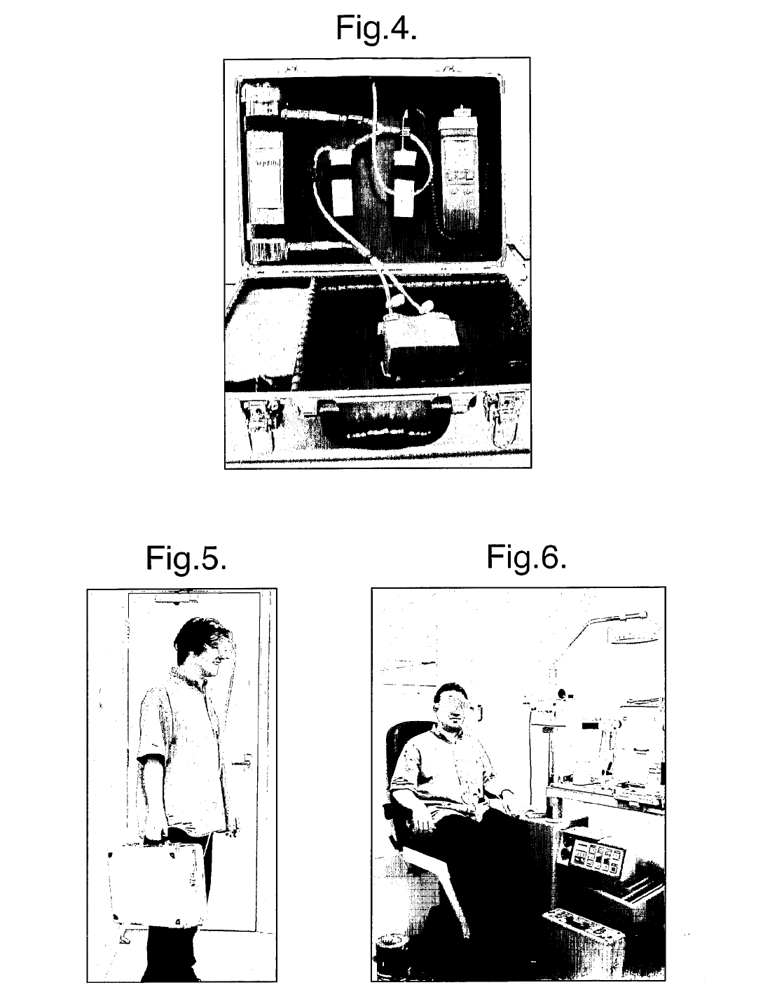

FIG. 4 is a picture of part of the apparatus of the present invention;

FIG. 5 is a picture of a user wearing the eye enclosure and illustrating the portability of the device; and

FIG. 6 is a picture of a user undergoing treatment using the apparatus of the present invention.

As illustrated in FIG. 1, the apparatus according to the first aspect of the present invention comprises an eye enclosure 1 which in this case is a pair of goggles. Air is drawn into the pump 2. Some of the air is passed in line 3 to a drying unit 4 and the remainder is passed in line 5 to a vessel 6 in which it is bubbled through a glycerol/water mixture. “Dry” air in line 7 and “wet” air in line 8 are joined via the Y-junction 9 and passed to a mixing chamber 10. A probe 11 is inserted into the mixing chamber and the humidity of the air measured by the meter 12 attached to the probe 11. The air exiting the mixing chamber is passed in line 13 to a Y-junction 14 where it is split into two lines 15 and 16 in which it is passed to the goggles 1 such that air is applied in the region of each eye. This portion of the apparatus is illustrated in detail in FIG. 2.

At least some of the elements of the apparatus may be placed within a carry case as illustrated in FIGS. 3 and 4. As illustrated in FIG. 5, the apparatus of the present invention enables the user to move freely whilst wearing the goggles. FIG. 6 illustrates a user using the apparatus in the optometrists office.

For the aspects of the present invention where it is desirable that the air supplied to the eye enclosure has a higher humidity than ambient air, the dry air line 7 and associated means for drying the air may be omitted. Similarly, where it is desirable that the air supplied to the eye enclosure has a lower humidity than ambient air, the wet air line 8 may be omitted. In one arrangement, both lines may be retained such that dry air may be mixed with air having been passed through the glycerol/water or water such that a particular level of increased humidity can be achieved.

The present invention will now be described with reference to the following examples.

### EXAMPLE 1

The baseline in vivo tear film was assessed by the measurement of the tear film non-invasive break up time (NIBUT), which is the time elapsed between eye opening after a blink and the destabilisation of the film. It is characterised by the appearance of the first dark spot within the tear film under wide diffuse light observation. The NIBUT measurements are recorded in seconds. Three consecutive measurements were carried out and the mean median and minimum values are calculated. The subjects then wore goggles in accordance with the present invention for 20 minutes at a relative humidity of 60% and the tear film evaluation was repeated. The subjects then wore the goggles for 10 minutes at a relative humidity of 1% and the tear film evaluated again. The results are set out in Table 1. Subject 1 is a contact lens wearer.

The results indicate a significant shortening of the tear break up time. That is to say that there is a significant destabilisation of the tear film after 10 min at 1% humidity. However, all subjects benefited from 20 min at 60% humidity with a significant increase in tear break up time. That is to say that a more stable tear film is obtained.

### EXAMPLE 2

A baseline in vivo tear film evaluation was carried out on a male subject 31 years of age. The subject then wore the goggles of the present invention for 10 minutes at a relative humidity of 85% and the tear film was evaluated again. The results are set out in Table 2.

### EXAMPLE 3

A baseline in vivo tear film evaluation was carried out on a male subject 31 years of age. The subject then wore the goggles of the present invention for 10 minutes at a relative humidity of 5% and the tear film was evaluated again. The results are set out in Table 3.

TABLE 1

| | Subject | Subject | Subject | Subject | Subject | Subject |
|---|---|---|---|---|---|---|
| | 1 right | 1 left | 2 right | 2 left | 3 right | 3 left |
| | eye | eye | eye | eye | eye | eye |
| Baseline | 8.8 | 7.5 | 12.5 | 14.9 | 14.8 | 14.7 |
| mean | | | | | | |
| Baseline | 9.0 | 7.6 | 13.5 | 14.8 | 13.5 | 14.8 |
| median | | | | | | |
| Baseline | 5.4 | 6.9 | 10.3 | 14.7 | 13.2 | 12.1 |
| minimum | | | | | | |
| 20 mins | 13.9 | 13.7 | 25.2 | 25.9 | 20.0 | 21.0 |
| at 60% | | | | | | |
| mean | | | | | | |
| 20 mins | 12.3 | 13.6 | 24.8 | 30.0 | 17.5 | 20.5 |
| at 60% | | | | | | |
| median | | | | | | |
| 20 mins | 12.0 | 13.4 | 24.1 | 17.6 | 16.8 | 20.1 |
| at 60% | | | | | | |
| minimum | | | | | | |
| 10 mins | 5.0 | 4.0 | 9.5 | 11.2 | 11.3 | 10.5 |
| at 1% | | | | | | |
| mean | | | | | | |
| 10 mins | 5.1 | 4.0 | 10.0 | 12.0 | 11.7 | 10.3 |
| at 1% | | | | | | |
| median | | | | | | |
| 10 mins | 4.5 | 3.2 | 8.0 | 8.3 | 10.0 | 7.8 |
| at 1% | | | | | | |
| minimum | | | | | | |

TABLE 2

| | Right Eye | Left Eye |
|---|---|---|
| Baseline mean | 17.0 | 39.4 |
| Baseline median | 13.0 | 44.8 |
| Baseline minimum | 12.4 | 23.4 |
| After 10 min at 85% mean | 41.0 | 47.6 |
| After 10 min at 85% median | 45.2 | 49.9 |
| After 10 min at 85% minimum | 27.9 | 43.1 |

TABLE 3

| | Right Eye | Left Eye |
|---|---|---|
| Baseline mean | 47.6 | 47.0 |
| Baseline median | 49.9 | 47.3 |
| Baseline minimum | 43.1 | 43.9 |
| After 10 min at 5% mean | 28.8 | 27.7 |
| After 10 min at 5% median | 29.7 | 31.4 |
| After 10 min at 5% minimum | 25.2 | 20.2 |

1-41. (canceled)

42. Apparatus which provides a dry environment around the eyes of the user comprising:
an eye enclosure adapted to provide an enclosed area about the eyes of the user;
means for retaining the eye enclosure in position; and
means for supplying dry air to the eye enclosure.

43. Apparatus according to claim 42 wherein the dry air will be air having a humidity of about 40% or less.

44. Apparatus according to claim 42 wherein the dry air is generated by passing the air through a container of a suitable desiccant or by passing the air across condensation coils.

45. Apparatus according to claim 42 wherein the dry air is pumped to the eye enclosure.

46. Apparatus according to claim 42 wherein the dry air provided to the eye enclosure is provided from a source of dry air and a source of wet air which are mixed such that a desired level of dryness can be achieved.

47. Apparatus according to claim 46 wherein the wet air is generated by passing the ambient air through water or a mixture of water and glycerol.

48. Apparatus according to claim 46 wherein the apparatus includes means for measuring the relative humidity of the mixed air present in, or supplied to, the enclosure, and means for adjusting the mixture so that the desired level of humidity may be achieved.

49. Apparatus according to claim 48 wherein the measurement means and the adjustment means allow the alteration of the humidity of the air supplied to the eye enclosure to be adjusted during operation.

50. Apparatus according to claim 48 wherein the wet and dry air is mixed in a mixing chamber before being supplied to the eye enclosure.

51. Apparatus according to claim 42 wherein the means for supplying dry air to the eye enclosure allows for substantially equal air flow to the region of each eye.

52. Apparatus according to claim 42 wherein the eye enclosure is a pair of goggles.

53. Apparatus according to claim 52, wherein the goggles have two chambers each to cover one eye.

54. Apparatus according to claim 52 wherein the goggles are made of plastics material.

55. Apparatus according to claim 42 wherein the apparatus is portable.

56. Apparatus according to claim 42 wherein the apparatus includes means for adjusting the temperature of the air which is present in, or supplied to, the eye enclosure.

57. A method of testing an item to be applied to the eye comprising:
applying the test item to at least one of the user's eyes;
subjecting the user to an environment around the eyes that is adjusted from the ambient environment by providing an eye enclosure and adapted to provide an enclosed area about the eyes of the user and supplying air having the adjusted environment to the eye enclosure; and monitoring the user.

58. A method of testing an item to be applied to the eye comprising:
subjecting the user to an environment around the eyes that is adjusted from the ambient environment by providing an eye enclosure adapted to provide an enclosed area about the eyes of the user and supplying air having the adjusted environment to the eye enclosure;
applying the test item to at least one of the user's eyes;
and monitoring the user.

59. The method according to claim 58 wherein the eye enclosure is removed before the test item is applied.

60. The method according to claim 59 wherein the eye enclosure is replaced once the test item has been applied to the at least one of the user's eyes.

61. The method according to claim 58 wherein the monitoring of the user is carried out periodically or continuously while the user is wearing the eye enclosure

62. The method according to claim 58 wherein the monitoring of the user is carried out once the subjection of the user to the chosen environment is completed or the user may periodically remove the eye enclosure for monitoring to take place.

63. A method of screening patients comprising the steps of:
subjecting the user to an environment around the eyes that is adjusted from the ambient environment by providing an eye enclosure and adapted to provide an enclosed area about the eyes of the user and supplying air having the adjusted environment to the eye enclosure;
and monitoring the user.

64. The method of claim 63 wherein the method is used to compare the performance of contact lens materials, contact lens care products and medicaments.

65. A method for diagnosing ocular and/or systemic medical conditions comprising:
subjecting the user to an environment around the eyes having an environment that is adjusted from the ambient environment by providing an eye enclosure and adapted to provide an enclosed area about the eyes of the user and supplying air having the adjusted environment to the eye enclosure; and monitoring the user.

66. The method according to claim 65 wherein the environment adjusted from the ambient environment has one or more of an adjusted humidity, an adjusted temperature and the addition of a foreign body.

67. The method according to claim 65 wherein the foreign body is pollen or pollutants.

68. The method according to claim 65 wherein the ocular condition is dry eye syndrome.

69. A method of treatment for patients having ocular and/or systemic medical condition comprising:
an eye enclosure adapted to provide an enclosed area about the eyes of the user;
means for retaining the eye enclosure in position; and
means for supplying air having a required humidity to the eye enclosure.

70. The method according to claim 69 wherein the ocular condition is dry eye syndrome.

71. The method according to claim 69 wherein the air provided to the eye enclosure has an increased humidity of at least 50%.

72. Apparatus for use in testing an item to be applied to the eye comprising:
an eye enclosure adapted to provide an enclosed area around the eyes of the user;
means for retaining the eye enclosure in position; and
means for supplying air to the eye enclosure having an environment that is adjusted from the ambient humidity.

73. Apparatus according to claim 72 wherein the test item is contact lens, contact lens material, contact lens care products, eye care products or medicaments.

74. Apparatus according to claim 72 wherein the test item is eye drops for use in the treatment of dry eye syndrome.

75. Apparatus for use in screening a patient comprising:
an eye enclosure adapted to provide an enclosed area around the eyes of the user;
means for retaining the eye enclosure in position; and
means for supplying air to the eye enclosure having a humidity that is adjusted from the ambient humidity.

76. Apparatus for use in diagnosing ocular and/or systemic medical conditions comprising:
an eye enclosure adapted to provide an enclosed area around the eyes of the user;
means for retaining the eye enclosure in position; and
means for supplying air to the eye enclosure having a humidity that is adjusted from the ambient humidity.

77. Apparatus according to claim 76 wherein the ocular condition is dry eye symptoms.

78. Apparatus for use in treating a patient suffering from symptoms of ocular and/or systemic medical condition comprising:
an eye enclosure adapted to provide an enclosed area around the eyes of the user;
means for retaining the eye enclosure in position; and
means for supplying air to the eye enclosure having a humidity that is adjusted from the ambient humidity.

79. Apparatus according to claim 78 wherein the environment is adjusted from the ambient environment has one or more of an adjusted humidity, an adjusted temperature and the addition of a foreign body.

80. Apparatus according to claim 79 wherein the foreign body is pollen or pollutants.
